(19) United States
(12) Patent Application Publication
(10) Pub. No.: US 2007/0240618 A1
(43) Pub. Date: Oct. 18, 2007

(54) High-Purity Naphthol as Pigments

(76) Inventors: Ulrike Rohr, Mannheim (DE); Ruediger Baur, Eppstein-Niederjosbach (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/578,739

(22) PCT Filed: Apr. 6, 2005
(86) PCT No.: PCT/EP05/03598

§ 371 Date: Apr. 4, 2007

(30) Foreign Application Priority Data
Apr. 22, 2004 (DE) 10 2004 019 560.9

(51) Int. Cl. C09B 62/01; C07C 245/08

(52) U.S. Cl. 106/496; 534/876

(57) ABSTRACT

Naphthol AS pigments of the formula (IV) 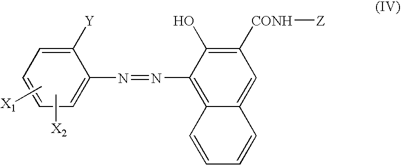 where X1, X2 Y and Z are as defined in the specification and have a maximum content of the secondary components (1) to (5) defined by the upper limits set forth in the table within the specification.

The present invention relates to the field of azo pigments.

Naphthol AS pigments are of particular industrial interest, since they usually attain high color strengths and cover the magenta region of the process ink set. They also have good lightfastnesses.

Naphthol AS pigments are traditionally produced in batch processes. These processes all require accurate policing of process parameters in that, for example, temperature, time, commixing and colorant concentration and the suspension concentration are decisive for the yield, the coloristic properties and the fastnesses of the pigments obtained and also for their consistency. Similarly, the scale-up of new products from the laboratory to manufacturing scale is costly and inconvenient for batch processes, and may cause difficulties, since, for example, tank and stirrer geometries or heat transfers have substantial influence on primary particle size, particle size distribution and coloristic properties.

Yet, despite all processing optimizations at synthesis, conventionally produced azo pigments do occasionally still contain, in their as-synthesized state, residual amounts of unconverted starting materials and also of by-products formed by secondary reactions.

Particularly those pigments used for non-impact printing processes, such as Small Office/Home Office printers, high chemical purity is an absolute prerequisite. For certain applications, such as the coloration of consumer articles for example, the colorants used have to meet specific limits for primary aromatic amines, naphthols and triazines.

It is an object of the present invention to provide Naphthol AS pigments containing a distinctly reduced level of undesirable secondary components.

The present invention accordingly provides Naphthol AS pigments of the formula (IV) 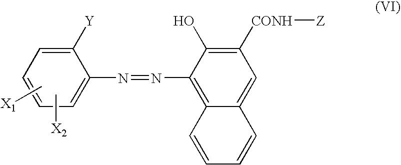
where
X₁ is hydrogen, halogen, nitro, carbamoyl, phenylcarbamoyl, sulfamoyl, phenylsulfamoyl, C₁-C₄-alkylsulfamoyl or di(C₁-C₄)-alkylsulfamoyl;
X₂ is hydrogen or halogen;
Y is hydrogen, halogen, nitro, C₁-C₄-alkyl, C₁-C₄-alkoxy or C₁-C₄-alkoxycarbonyl; and

Z is phenyl, naphthyl, benzimidazolonyl, phenyl or halogen-, nitro-, C₁-C₄-alkyl- and/or C₁-C₄-alkoxy-substituted phenyl, having a maximum content of hereinbelow specified secondary components (1) to (5), defined by the following upper limits:

| | Secondary component: | | Upper limit: |
|---|---|---|---|
| 1 | Amine of formula | H₂N—Ar | 100 ppm |
| 2 | Amine of formula | H₂N—Z | 50 ppm |
| 3 | Triazene of formula | Ar—N═N—N—Ar | 50 ppm |
| 4 | Mixed triazene of formula | Ar—N═N—NHZ | 50 ppm |

| | | |
|---|---|---|
| 5 | 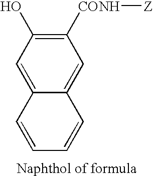 | 400 ppm |

where Ar has the meaning 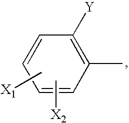
each determined by high pressure liquid chromatography (HPLC).

Preference for the purposes of the present invention is given to Naphthol AS pigments of the formula (IV) having a content of not more than 80 ppm and in particular not more than 60 ppm for secondary component 1.

Preference for the purposes of the present invention is given to Naphthol AS pigments of the formula (IV) having a secondary component 2 content below the detection limit of 50 ppm.

Preference for the purposes of the present invention is given to Naphthol AS pigments of the formula (IV) having a secondary component 3 content below the detection limit of 50 ppm.

Preference for the purposes of the present invention is given to Naphthol AS pigments of the formula (IV) having a secondary component 4 content below the detection limit of 50 ppm.

Preference for the purposes of the present invention is given to Naphthol AS pigments of the formula (IV) having a content of not more than 200 ppm and in particular not more than 100 ppm for secondary component 5.

The secondary components (1) to (5) can be formed as follows:
(1): by scissioning of the diazo compound used;
(2): by scissioning the amide bond of the coupler used;
(3): from the diazo compound and the amine (1) which was released as described above;
(4): from the diazo compound and the amine (2) which was released as described above;
(5): is unconverted coupler.

To determine the secondary components by HPLC, a sample of the compound of formula (IV) (each sample 0.5 g) is dried, suspended with N-methylpyrrolidone and methanol and the filtrate is analyzed via an HPLC system equipped with UV-Vis detector.

Preference for the purposes of the present invention is given to high-purity Naphthol AS pigments of the formula (IV) where
Y is hydrogen, methoxy, methoxycarbonyl, methyl or chlorine;
X₁ is at position 5 and is hydrogen, chlorine, nitro, carbamoyl, phenyl-carbamoyl, sulfamoyl, phenylsulfamoyl, methylsulfamoyl or dimethylsulfamoyl;
X₂ is at position 4 and is hydrogen or chlorine; and
Z is a chlorine-, nitro-, C₁-C₂-alkyl- and/or C₁-C₂-alkoxy-substituted phenyl.

Particular preference for the purposes of the present invention is given to the pigments C.I. Pigment Red 146, 147, 176, 184, 185, 269.

The present invention also provides a process for producing such high-purity Naphthol AS pigments, which comprises
(a) conducting at least the azo coupling in a microreactor,
(b) intensively contacting the Naphthol AS pigment produced in the microreactor with an organic solvent selected from the group of the C₃-C₆ alcohols, the C₄-C₁₀ ether alcohols and the halogenated aromatics at 0 to 60° C., and/or
(c) subjecting the Naphthol AS pigment produced in the microreactor to a membrane purification in aqueous or solvent-containing suspension.

Step (c) can also be performed before step (b). It may also be possible in some cases that the desired degree of purity is already achieved through one of the steps (b) or (c).

(a) Synthesis in Microreactor:

Useful microreactors include the apparatuses described in WO 01/59013 A1. A microreactor is constructed from a plurality of laminae which are stacked and bonded together and whose surfaces bear micromechanically created structures which cooperate to form reaction spaces for chemical reactions. The system contains at least one continuous channel connected to the inlet and the outlet. The flow rates of the streams of material are limited by the apparatus, for example by the pressures which result depending on the geometry of the microreactor. It is desirable for the microreactor reaction to go to completion, but it is also possible to adjoin a delay zone to create a delay time that may be required. The flow rates are advantageously between 0.05 and 5 l/min, preferably between 0.05 and 500 ml/min, more preferably between 0.05 and 250 ml/min and especially between 0.1 and 100 ml/min.

The microreaction system is operated continuously, and the quantities of fluid which are mixed with each other are in the microliter (μl) to milliliter (ml) range. The dimensions of the microstructured regions within the reactor are decisive for the production of Naphthol AS pigment in this microreaction system. These dimensions have to be sufficiently large that, in particular, solid particles can pass without problem and so not clog up the channels. The smallest clear width of the microstructures should be about ten times larger than the diameter of the largest pigment particles. Furthermore, it has to be ensured, through appropriate geometric styling, that there are no dead water zones, for example dead ends or sharp corners, where pigment particles for example can sediment. Preference is therefore given to continuous paths having round corners. The structures have to be sufficiently small to exploit the intrinsic advantages of microreaction technology, namely excellent thermal control, laminar flow, diffusive mixing and low internal reaction volume.

The clear width of the solution- or suspension-ducting channels is advantageously 5 to 10 000 μm, preferably 5 to 2000 μm, more preferably 10 to 800 μm, especially 20 to 700 μm.

The clear width of the heat exchanger channels depends primarily on the clear width of the liquid- or suspension-ducting channels and is advantageously not more than 10 000 μm, preferably not more than 2000 μm and especially not more than 800 μm. The lower limit of the clear width of the heat exchanger channels is uncritical and is at most constrained by the pressure increase of the heat exchanger fluid to be pumped and by the necessity for optimal heat supply or removal.

The dimensions of the microreaction system used are:

| | |
|---|---|
| heat exchanger structures: | channel width about 600 μm, channel height: |
| | about 250 μm; |
| mixer and delay time: | channel width about 600 μm, channel height |
| | about 500 μm. |

The microreactor is preferably charged with all heat exchanger fluids and reactants from above. The product and the heat exchanger fluids are also preferably removed upwardly. The possible supply of third and fourth liquids involved in the reaction (buffer solutions being an example) is realized via a T-junction located directly upstream of the reactor, i.e., one reactant at a time can be mixed with the buffer solution in advance. The requisite concentrations and flows are preferably policed via precision piston pumps and a computer-controlled control system. The reaction temperature is monitored via integrated sensors and monitored and controlled with the aid of the control system and of a thermostat/cryostat.

The preparation of mixtures of input materials can also be carried out in advance in micromixers or in upstream mixing zones. It is also possible for input materials to be metered into downstream mixing zones or into downstream micromixers or -reactors.

The system used here is made of stainless steel; other materials, for example glass, ceramic, silicon, plastics or other metals, are similarly useful.

As well as the azo coupling, the diazotization, can also be carried out in the microreactor. It is also possible to carry out both stages in microreactors connected in series.

It is advantageous to supply the reactants to the microreactor as aqueous solutions or suspensions and preferably in stoichiometric/equivalent amounts. The azo coupling reaction takes place preferably in aqueous solution or suspension, although it is also possible to use organic solvents, alone or as a mixture with water; by way of example, alcohols having from 1 to 10 carbon atoms, examples being methanol, ethanol, n-propanol, isopropanol, butanols, such as n-butanol, sec-butanol, and tert-butanol, pentanols, such as n-pentanol and 2-methyl-2-butanol, hexanols, such as 2-methyl-2-pentanol, 3-methyl-3-pentanol, 2-methyl-2-hexanol and 3-ethyl-3-pentanol, octanols, such as 2,4,4-tri-methyl-2-pentanol, and cyclohexanol; or glycols, such as ethylene glycol, diethylene glycol, propylene glycol, dipropylene glycol, or glycerol; polyglycols, such as polyethylene glycols or polypropylene glycols; ethers, such as methyl isobutyl ether, tetrahydrofuran or dimethoxyethane; glycol ethers, such as monomethyl or monoethyl ethers of ethylene glycol or propylene glycol, diethylene glycol monomethyl ether, diethylene glycol monoethyl ether, butyl glycols or methoxybutanol; ketones, such as acetone, diethyl ketone, methyl isobutyl ketone, methyl ethyl ketone or cyclohexanone; aliphatic acid amides, such as formamide, dimethylformamide, N-methylacetamide or N,N-dimethylacetamide; urea derivatives, such as tetramethylurea; or cyclic carboxamides, such as N-methyl-pyrrolidone, valerolactam or caprolactam; esters, such as carboxylic acid C₁-C₆ alkyl esters, such as butyl formate, ethyl acetate or propyl propionate; or carboxylic acid C₁-C₆ glycol esters; or glycol ether acetates, such as 1-methoxy-2-propyl acetate; or phthalic or benzoic acid C₁-C₆ alkyl esters, such as ethyl benzoate; cyclic esters, such as caprolactone; nitriles, such as acetonitrile or benzonitrile; aliphatic or aromatic hydrocarbons, such as cyclohexane or benzene; or alkyl-, alkoxy-, nitro- or halo-substituted benzene, such as toluene, xylenes, ethylbenzene, anisole, nitrobenzene, chlorobenzene, o-dichlorobenzene, 1,2,4-trichlorobenzene or bromobenzene; or other substituted aromatics, such as benzoic acid or phenol; aromatic heterocycles, such as pyridine, morpholine, picoline or quinoline; and also hexamethylphosphoramide, 1,3-dimethyl-2-imidazolidinone, dimethyl sulfoxide, and sulfolane. Said solvents may also be used as mixtures. Preference is given to using water-miscible solvents.

The process of the present invention may also utilize the auxiliaries that are employed in conventional processes, for example surfactants, pigmentary and nonpigmentary dispersants, fillers, standardizers, resins, waxes, defoamers, antidust agents, extenders, shading colorants, preservatives, drying retardants, rheology control additives, wetting agents, antioxidants, UV absorbers, photostabilizers or a combination thereof.

The auxiliaries may be added at any point in time before, during or after the reaction in the micro reactor, all at once or in two or more portions. The auxiliaries may, for example, be added directly to the reactant solutions or suspensions, or else during the reaction in liquid, dissolved or suspended form.

The overall amount of the added auxiliaries may amount to from 0 to 40% by weight, preferably from 1 to 30% by weight, more preferably from 2.5 to 25% by weight, based on the Naphthol AS pigment.

Suitable surfactants include anionic or anion-active, cationic or cation-active, and nonionic substances or mixtures of these agents.

Examples of surfactants, pigmentary and nonpigmentary dispersants which can be used for the method of the invention are specified in EP-A-1 195 411.

Since compliance with a desired pH value during and after the reaction is often decisive for quality, it is also possible to supply buffer solutions, preferably of organic acids and salts thereof, such as formic acid/formate buffers, acetic acid/acetate buffers, citric acid/citrate buffers; or of inorganic acids and salts thereof, such as phosphoric acid/phosphate buffers or carbonic acid/hydrogencarbonate or carbonate buffers, for example.

(b) Solvent Wash:

The solvent wash of the present invention comprises the take-up in one of the organic solvents mentioned of the Naphthol AS pigment prepared in step (a), either directly from the microreactor or after intervening isolation for example as a presscake (solids content about 5% to 30% by weight).

Preferred solvents here are C₃-C₄ alcohols, glycol ethers and chlorinated benzenes, for example butoxyethanol, orthodichlorobenzene, isobutanol, isopropanol, or a mixture thereof.

It is also possible to use a pigment suspension treated as per (c).

The amount of solvent is preferably in the range from 1% to 30% by volume and in particular in the range from 5% to 15% by volume, based on the volume of the pigment suspension, or 1 to 10 times the weight of solvent, based on the weight of the pigment in the presscake.

The mixture of pigment suspension or presscake and solvent is preferably stirred at between 10° C. and 50° C. and especially between 20° C. and 45° C. for preferably 0.1 to 2 hours and especially 0.25 to 1 hour and preferably at atmospheric pressure.

Normal stirring apparatus can be used, such as laboratory stirrers for example. However, it is also possible in principle to use an inline dispersing machine fitted with appropriate dispersing tools, in the pumped circulation system of the feed vessel. Such a dispersing machine not only ensures an intensive commixing of the suspension in the feed vessel, but also has a deagglomerating effect, so that any inclusions are laid bare.

The solvent-treated pigment suspension is subsequently filtered and washed or fed to the membrane purification stage (c).

(c) Membrane Purification:

The membrane purification stage of the present invention comprises passing an azo colorant suspension obtained from step (a) or from (b) through a membrane system constituted such that the Naphthol AS pigment is held back by the membrane as completely as possible. The liquid medium can be in particular water or else an organic solvent, if appropriate in admixture with water. The solids concentration in the suspension is advantageously in the range from 1% to 10% by weight and preferably in the range from 2% to 5% by weight, based on the total weight of the suspension. The driving force for transmembrane transport is a pressure difference between the two sides of the membrane. The pressure difference is advantageously in the range from 0.5 to 5 bar and preferably in the range from 1 to 2 bar. The pressure is generated by suitable pumps for example, examples being piston pumps. The membranes used are for example ceramic or polymeric membranes having typical separation limits between 100 and 10⁶ g/mol. Preference is given to using static membrane modules, such as tubular or plate modules, or dynamic membrane modules. The temperature is advantageously in the range from 0 to 100° C. and particularly within the range from 20 to 80° C.

The membrane purification can also be carried out as a diafiltration. In this case, the retentate, i.e., the azo pigment, is recycled into the original vessel and the water or solvent content is kept constant by replenishment. The process of the present invention provides the following product improvements compared with a traditional optimized batch operation:

Step (a) lowers the level of triazene and mixed triazenes significantly, i.e., down to below the detection limit of 50 ppm, but over 100 ppm of free aromatic amine H₂N—Ar and of unconverted coupling components, for example naphthol, are usually still present.

Step (b) or step (c), preferably step (b) combined with step (c), surprisingly provides a lowering of the free amine and naphthol content often below the detection limits of 25 ppm and 100 ppm, respectively.

Inorganic salts are likewise retained as a side effect of membrane purification.

The high-purity Naphthol AS pigments according to the present invention are used in particular for coloration of electrophotographic toners and developers, for example one- or two-component powder toners (also known as one- or two-component developers), magnetic toners, liquid toners, latex toners, addition polymerization toners and also specialty toners, of powder coatings, of ink jet inks and color filters and also as colorants for electronic inks (“e-inks”) or electronic paper (“e-paper”).

Toner particles can also be used for cosmetic and pharmaceutical applications, e.g. for coating tablets.

Typical toner binders are addition polymerization, polyaddition and polycondensation resins, such as styrene, styrene-acrylate, styrene-butadiene, acrylate, polyester, phenol-epoxy resins, polysulfones, polyurethanes, individually or in combination, and also polyethylene and polypropylene, which may each contain further ingredients, such as charge control agents, waxes or flow assistants, or are subsequently modified with these additives.

The Naphthol AS pigments of the present invention are obviously also very generally useful for pigmentation of macromolecular organic materials of natural or synthetic origin, in particular of plastics, resins, coatings, paints, electrophotographic toners and developers, electret materials, color filters and also of inks, including printing inks, and seed.

Macromolecular organic materials pigmentable with the Naphthol AS pigments of the present invention are for example cellulose compounds, such as cellulose ethers and cellulose esters, for example ethylcellulose, nitrocellulose, cellulose acetates or cellulose butyrates, natural binders, for example fatty acids, fatty oils, resins and their conversion products, or artificial resins, such as polycondensates, polyadducts, addition polymers and addition copolymers, for example amino resins, in particular urea- and melamine-formaldehyde resins, alkyd resins, acrylic resins, phenoplasts and phenolic resins, such as novolaks or resoles, urea resins, polyvinyls, such as polyvinyl alcohols, polyvinyl acetals, polyvinyl acetates or polyvinyl ethers, polycarbonates, polyolefins, such as polystryene, polyvinyl chloride, polyethylene or polypropylene, poly(meth)acrylates and their copolymers, such as polyacrylic esters or polyacrylonitriles, polyamides, polyesters, polyurethanes, coumarone-indene and hydrocarbon resins, epoxy resins, unsaturated synthetic resins (polyesters, acrylates) having different curing mechanisms, waxes, aldehydic and ketonic resins, gum, rubber and its derivatives and latices, casein, silicones and silicone resins; individually or in mixtures. It is immaterial whether the macromolecular organic compounds mentioned are present as plastically deformable masses, melts or in the form of spinning solutions, dispersions, lacquers, paints or printing inks.

Depending on the planned use, it is advantageous to use the Naphthol AS pigments of the present invention as a blend or in the form of formulations or dispersions. Based on the macromolecular organic material to be pigmented, the Naphthol AS pigments of the present invention are used in an amount of 0.05% to 30% by weight and preferably 0.1% to 15% by weight.

For applications where high-purity pigments are not needed but certain purity criteria have to be fulfilled nonetheless, it can be economically sensible to blend the Naphthol AS pigments of the present invention with conventionally produced Naphthol AS pigments such that the stipulated degrees of purity are still fulfilled. It is also possible in some cases to use in lieu of a ground and/or finished Naphthol AS pigment of the present invention a corresponding crude having a BET surface area of greater than 2 m²/g and preferably greater than 5 m²/g. This crude can be used for producing color concentrates in liquid or solid form in concentrations of 5% to 99% by weight, alone or if appropriate admixed with other crudes or ready-produced pigments.

The present invention further provides for the use of the colorant formulation described as a colorant for jettable printing inks, in particular for ink jet inks. Ink jet inks refers not only to inks on an aqueous basis (including microemulsion inks) and on a nonaqueous basis (solvent-based), UV-curable inks but also to such inks as operate by the hot melt process.

Solvent-based ink jet inks contain essentially 0.5% to 30% by weight and preferably 1% to 15% by weight of one or more of the Naphthol AS pigments of the present invention, 70% to 95% by weight of an organic solvent or solvent mixture and/or of a hydrotropic compound. If appropriate, the solvent-based ink jet inks can contain carrier materials and binders which are soluble in the solvent, examples being polyolefins, natural rubber, synthetic rubber, polyvinyl chloride, vinyl chloride-vinyl acetate copolymers, poly(vinyl butyral)s, wax-latex systems or combinations thereof.

If appropriate, solvent-based ink jet inks may include further additives, examples being wetting agents, degassers/defoamers, preservatives and antioxidants. Microemulsion inks are based on organic solvents, water and if appropriate an additional substance (surfactant) which acts as an interfacial mediator. Microemulsion inks contain 0.5% to 30% by weight and preferably 1% to 15% by weight of the Naphthol AS pigments of the present invention, 0.5% to 95% by weight of water and 0.5% to 95% by weight of organic solvents and/or interfacial mediators.

UV-curable inks contain essentially 0.5% to 30% by weight of the Naphthol AS pigments of the present invention, 0.5% to 95% by weight of water, 0.5% to 95% by weight of an organic solvent or solvent mixture, 0.5% to 50% by weight of a radiation-curable binder and if appropriate 0% to 10% by weight of a photoinitiator.

Hot melt inks are usually based on waxes, fatty acids, fatty alcohols or sulfonamides which are solid at room temperature and liquefy on heating, the preferred melting range being between about 60 and about 140° C.

Hot melt ink jet inks consist essentially of 20% to 90% by weight of wax and 1% to 10% by weight of the Naphthol AS pigments of the present invention. They may further include 0% to 20% by weight of an additional polymer (as “dye dissolver”), 0% to 5% by weight of dispersant, 0% to 20% by weight of viscosity modifier, 0% to 20% by weight of plasticizer, 0% to 10% by weight of tackifying additive, 0% to 10% by weight of transparency stabilizer (prevents crystallization of waxes, for example) and also 0% to 2% by weight of an antioxidant.

The present invention's printing inks, especially ink jet inks, can be produced by dispersing the Naphthol AS pigment into the microemulsion medium, into the nonaqueous medium or into the medium for producing the UV-curable ink or into the wax for producing a hot melt ink jet ink.

Advantageously, the as-obtained printing inks for ink jet applications are subsequently filtered, for example through a 1 μm filter.

The Naphthol AS pigments of the present invention are further useful as a colorant for color filters, not only for additive but also for subtractive color generation, and also as a colorant for electronic inks (“e-inks”) or electronic paper (“e-paper”).

To produce color filters, not only reflecting but also transparent color filters, pigments are applied in the form of a paste or as a pigmented photoresist in a suitable binder (acrylates, acrylic esters, polyimides, polyvinyl alcohols, epoxides, polyesters, melamines, gelatin, caseins) to the respective LCD components (e.g. TFT-LCD=Thin Film Transistor Liquid Crystal Displays or for example ((S) TN-LCD=(Super) Twisted Nematic-LCD). As well as a high thermal stability, a high pigment purity is a prerequisite for a stable paste or a pigmented photoresist. In addition, the pigmented color filters can also be applied by ink jet printing processes or other suitable printing processes.

### EXAMPLE 1

### C.I. Pigment Red 269

a1) Preparation of an Anisbase Diazonium Salt Solution:

242 g of 3-amino-4-methoxybenzanilide are initially stirred homogeneously into an initial charge of 2532 g of water at room temperature, precipitated by addition of hydrochloric acid and cooled down to 10° C. with 1.5 kg of ice/water. The precipitated hydrochloride is diazotized with 138 ml of sodium nitrite solution (40%) to finally give a readily stirrable anisbase diazo solution. This solution has a clarifying aid added to it and is subsequently filtered off into a feed vessel. Excess nitrite is removed by addition of amidosulfonic acid.

a2) Preparation of a Buffer for the Anisbase Diazonium Salt Solution:

To an initial charge of 1884 g of ice/water are added 502 g of acetic acid and also 614 g of aqueous sodium hydroxide solution, and the temperature is held at room temperature after addition of 1 kg of water.

a3) Preparation of a Solution of the Coupling Component (Naphthol AS-CA):

An initial charge of 2720 g of water containing a wetting aid is heated to 80° C. While stirring, 328 g of N-(5-chloro-2-methoxyphenyl)-3-hydroxynaphthalene-2-carboxamide are introduced and dissolved alkalinically. By addition of a further 2720 g of ice/water, the Naphthol AS solution is cooled down to room temperature. It is finally filtered by addition of a clarifying aid.

a4) Azo Coupling in Microreactor:

The anisbase diazonium salt solution and the Naphthol AS solution are pumped at a flow rate of 8 ml/min into the respective reactant inlets of the microreactor (type: Cytos from CPC-Systems/Frankfurt). To achieve the requisite pH of 4.8-5.0 for azo coupling, the reactant solutions are diluted with the acetic acid/acetate buffer prepared according to a2), shortly upstream of the reactor inlets. The buffer solution is likewise conveyed with the aid of calibrated piston pumps via a T-junction into the reactant feed lines of the microreactor at a flow rate of 6 ml/min in each case. The heat exchanger circuit of the microreactor is connected to a thermostat which sets the desired reaction temperature of 20° C. to 35° C. The coupled pigment suspension (21° C., pH=5.0) is collected in a feed vessel and subjected to the following solvent wash.

b) Solvent Wash:

The pigment suspension obtained from the microreactor is admixed with such an amount of butoxyethanol that the entire slurry contains about 10% by volume of butoxyethanol. The slurry is stirred at about 45° C. for 30 minutes, filtered off and washed with water. After sampling, the colorant-solvent-water suspension is subjected to the following membrane purification.

c) Membrane Purification:

A ceramic multichannel microfiltration membrane having a nominal separation limit of 60 nm for the separation-selective layer and a membrane area of 0.09 m² is used. About 15 kg of the colorant suspension having a pigment content of about 2% by weight are charged to a temperature-controllable feed vessel. The membrane is subjected to a pressure of about 1.5 bar on the retentate side at ambient temperature. To ensure a constant volume in the feed vessel, the mass of permeate removed is replaced with demineralized water in a discontinuous manner.

The pigment is fully retained and the organic secondary components are reduced to the values listed in table 2, under these conditions. The exchange volume (i.e., volume of demineralized water supplied/volume of pigment suspension used) is about 4. Permeate flux is about 200 l/(m²*h*bar).

At the same time, the initial chloride ion content of 2.5% is reduced by 10 hours of diafiltration to 920 ppm as is the sulfate content from initially 0.3% to 30 ppm.

d) Analysis:

The samples taken (each 0.5 g) are dried, admixed with 10 ml each of N-methylpyrrolidone and comminuted for 15 min by ultrasonication. After addition of 20 ml of methanol and renewed grinding for 15 min, the suspension is filtered off. In each case, 20 μl of the filtrate are introduced into the autosampler of the HPLC system and detected by UV-V is detector at 240 and 375 nm (separating column Nucleosil 120-5 C18 (length: 25 cm, Øᵢ=4.6 mm); mobile phase consisting of a buffer (575 mg of NH₄H₂PO₄ plus 1000 g of H₂O plus 3.0 g of NaN₃ (pH 5.0)) and methanol ®Chromasolv in various compositions for a total flux of 1 ml/min).

Table 2 lists the levels of secondary components after each step:

Table 2 shows a comparison of the typical secondary component levels of the conventional batch pigment with the secondary component levels of the pigment from a synthesis in a microreactor [step (a)] and subsequent solvent wash [step (b)] and membrane purification [step (c)].

The detection limits for the secondary components considered are listed in table 1 to categorize and assess the values in table 2. The measuring accuracy of the analytical method chosen is about ±5 ppm.

TABLE 1
Detection limits for secondary components:

| Secondary component | Detection limit |
|---|---|
| Anisbase, e.g. 3-amino- | 25 ppm |
| 4-methoxybenzanilide | |
| Chloromethoxyaniline | 50 ppm |
| Anisbase triazene | 50 ppm |
| Mixed triazene | 50 ppm |
| Naphthol AS-CA | 100 ppm |

TABLE 2
Comparison of secondary component levels in pigment from batch
synthesis versus microreactor synthesis with subsequent
solvent wash and membrane purification.

| | | | Pigment | Pigment |
|---|---|---|---|---|
| | | | after | after |
| | | Pigment | solvent | membrane |
| | Batch | after | wash | purification |
| | pigment | [step a)] | [step b)] | [step c)] |
| 3-Amino-4- | 132 ppm | 100 ppm | 80 ppm | 60 ppm |
| methoxybenzanilide | | | | |
| Chloromethoxyaniline | 54 ppm | 50 ppm | n.d.* | n.d.* |
| Anisbase triazene | 134 ppm | n.d.* | n.d.* | n.d.* |
| Mixed triazene | 138 ppm | n.d.* | n.d.* | n.d.* |
| Naphthol AS-CA | 500 ppm | <100 ppm | <100 ppm | <100 ppm |

*not detectable, i.e., smaller than detection limit of table 1.

### EXAMPLE 2

### C.I. Pigment Red 146

Steps a)-d) are carried out similarly to Example 1. The pigment obtained after step c) had anisbase, chloromethoxyaniline, anisbase triazene and Naphtol AS levels below the respective limit of detection.

### EXAMPLE 3

### C.I. Pigment Red 147

Steps a)-d) are carried out similarly to Example 1. The pigment obtained after step c) had anisbase, chloromethoxyaniline, anisbase triazene and Naphtol AS levels below the respective limit of detection.

### COMPARATIVE EXAMPLES 2 AND 3

Average values of altogether 80 batch syntheses:

| Anisbase | 519 ppm |
|---|---|
| Chloromethoxyaniline | 32 ppm |
| Anisbase triazene | 446 ppm |
| Naphtol AS | 1.10% |

1) A Naphthol AS pigment of the formula (IV) 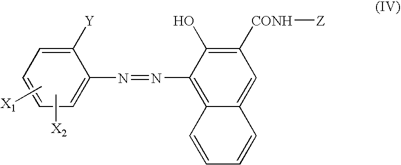
wherein
X₁ is hydrogen, halogen, nitro, carbamoyl, phenylcarbamoyl, sulfamoyl, phenylsulfamoyl, C₁-C₄-alkylsulfamoyl or di(C₁-C₄)-alkylsulfamoyl;
X₂ is hydrogen or halogen;
Y is hydrogen, halogen, nitro, C₁-C₄-alkyl, C₁-C₄-alkoxy or C₁-C₄-alkoxy-bonyl; and
Z is phenyl, naphthyl, benzimidazolonyl, phenyl or halogen-, nitro-, C₁-C₄-alkyl- and/or C₁-C₄-alkoxy-substituted phenyl,
having a maximum content of hereinbelow specified secondary components (1) to (5), defined by the following upper limits:

| | Secondary component: | | Upper limit: |
|---|---|---|---|
| 1 | Amine of formula | H₂N—Ar | 60 ppm |
| 2 | Amine of formula | H₂N—Z | 50 ppm |
| 3 | Triazene of formula | Ar—N═N—N—Ar | 50 ppm |
| 4 | Mixed triazene of formula | Ar—N═N—NHZ | 50 ppm |
| | | |
|---|---|---|
| 5 | 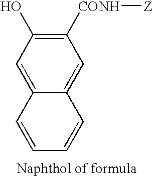 | 200 ppm |

wherein Ar has the meaning 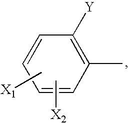
each determined by high pressure liquid chromatography.

2) A pigment as claimed in claim 1, having a content of not more than 100 ppm for secondary component (1).

3) A pigment as claimed in claim 1, wherein,
Y is hydrogen, methoxy, methoxycarbonyl, methyl or chlorine;
X₁ is at position 5 and is hydrogen, chlorine, nitro, carbamoyl, phenylcarbamoyl, sulfamoyl, phenylsulfamoyl, methylsulfamoyl or dimethylsulfamoyl;
X₂ is at position 4 and is hydrogen or chlorine; and
Z is a chlorine-, nitro-, C₁-C₂-alkyl- and/or C₁-C₂-alkoxy-substituted phenyl

4)-6) (canceled)

7) A pigment as claimed in claim 1, wherein the pigment is selected from the group consisting of C.I. Pigment Reds 146, 147, 176, 184, 185 and 269.

8) A process for producing a Naphthol AS pigment according to claim 1, comprising the steps of
(a) conducting at least the azo coupling in a microreactor,
(b) intensively contacting the Naphthol AS pigment produced in the microreactor with an organic solvent at a temperature of 0 to 60° C., wherein the organic solvent is selected from the group consisting of C₃-C₆ alcohols, C₄-C₁₀ ether alcohols and halogenated aromatics, and/or
(c) subjecting the Naphthol AS pigment produced in the microreactor to a membrane purification in aqueous or solvent-containing suspension.

9) A macromolecular organic material of natural or synthetic origin, comprising a Naphthol AS pigment as claimed in claim 1, wherein the macromolecular organic material of natural or synthetic origin is selected fro the group consisting of plastics, resins, coatings, paints, electrophotographic toners, electrophotographic developers, electret materials, color filters, inks, printing inks, and seed.

10) A pigmented composition comprising a Naphthol AS pigment as claimed in claim 1, wherein the composition is selected from the group consisting of one or two component powder toners, magnetic toners, liquid toners, addition polymerization toners, ink jet inks, in color filters electronic inks and electronic paper.
